(19) United States
(12) Patent Application Publication
(10) Pub. No.: US 2020/0046479 A1
(43) Pub. Date: Feb. 13, 2020

(54) NOVEL INCISION-FREE ADAPTIVE TVT-XO FOR TREATMENT OF URINARY INCONTINENCE

(71) Applicant: THE FIRST AFFILIATED HOSPITAL OF WENZHOU MEDICAL UNIVERSITY, Wenzhou (CN)

(72) Inventors: Haihong JIANG, Wenzhou (CN); Huiliang WANG, Wenzhou (CN); Shi LI, Wenzhou (CN); Lingxiao JI, Wenzhou (CN); Meihao WANG, Wenzhou (CN)

(21) Appl. No.: 16/585,490

(22) Filed: Sep. 27, 2019

Related U.S. Application Data
(63) Continuation of application No. PCT/CN2018/077567, filed on Feb. 28, 2018.

(30) Foreign Application Priority Data
Mar. 29, 2017 (CN) 201710195362.4

(51) Int. Cl. A61F 2/00

(52) U.S. Cl. CPC A61F 2/0031; A61F 2220/0008; A61F 2250/0007

(57) ABSTRACT

The present invention relates to a novel incision-free adaptive TVT-XO for treatment of urinary incontinence, comprising a tape body (1), wherein the tape body (1) comprises a left tape (11) and a right ape (12); each of the left tape (11) and the right ape (12) comprises a mesh band (13); a fixing anchor (14), configured to fix one end of the tape body (1) between the obturator fascia and the obturator externus muscle of the pelvis, is disposed at one end of the mesh band (13); male and female snap buckles (15) are disposed at the other ends of the mesh bands (13) of both the left tape (11) and the right ape (12), respectively; the left tape (11) and the right ape (12) are combined by means of insertion of the male and female snap buckles (15) to form a cross structure, which is “ [symbol] ”-shaped.

### CROSS-REFERENCE TO RELATED APPLICATIONS

This application is a continuation of International Patent Application No. PCT/CN2018/077567, filed on Feb. 28, 2018, which claims the benefit of priority from Chinese Patent Application No. 201710195362.4, filed on Mar. 29, 2017. The contents of the aforementioned application, including any intervening amendments thereto, are incorporated herein by reference in its entirety.

### TECHNICAL FIELD

The present invention relates to medical instruments and in particular to a novel incision-free adaptive TVT-XO for treatment of urinary incontinence.

### BACKGROUND OF THE PRESENT INVENTION

At present, among the implantable synthetic tapes for surgical treatment of urinary incontinence, the most commonly used is the mid-urethral sling operation which is the standard method for surgical treatment of female stress urinary incontinence. Although various tape systems involved are different in surgical approaches and access methods and also slightly different in specific designs, the overall principle is to implant, by a guide handle, a thin synthetic tape into a tissue space between the suburethra and the anterior vaginal wall. This can correct and reduce the oscillation of the urethra, so that it can be relatively fixed to realize a certain degree of anatomical reduction. On the other hand, the implanted mesh tape plays a flexible suspension and support role through certain viscosity and tissue reaction with the tissue, similarly to a hammock. In this way, it is possible to prevent the urinary leakage as the abdominal stress increases when a person coughs, jumps, and does physical activities. At present, the materials for synthetic tapes are generally meshed polymers which are non-absorbable or slowly absorbable, such as polypropylene. During the postoperative healing process, the fusion between the tape and the tissue is strengthened by the reaction between the synthetic material and the tissue, so the support and fixation function is strengthened. Therefore, generally, for the common urinary incontinence, the synthetic tape surgery has a very high success rate in short and even medium term. However, the synthetic tapes and accessories commonly used in clinical practice are not applicable to some types of stress urinary incontinence, especially very severe urinary incontinence accompanied with weak contractility of the urethral sphincter muscle, or also accompanied with weak contractility of the bladder detrusor muscle. Those complex and refractory types of urinary incontinence often seriously affect the quality of life. Continuous urinary leakage may lead to incontinence-associated dermatitis, and may greatly increase urinary tract infections, thus aggravating a series of lower urinary tract symptoms during urine storage and micturition and also deteriorating the function of the damaged bladder.

The existing tape is designed to one-time suspend the urethra physically in one direction. Depending upon different surgical approaches and tape suspension directions, the urethra with great oscillation can be corrected to different degrees to restore, as much as possible, the function of the urethra against the urinary leakage caused by the abdominal stress on the bladder, in order to improve urinary incontinence. At present, at least one or more incisions may be caused by the implantation of the tape, and after the tape is fixed, it is difficult to manually adjust the tape directly after the operation. Moreover, a too tight or too loose tape may lead to dysuria or lack of significant improvement to urinary incontinence. In this case, it is often necessary to take targeted measures or adjust the tape by another operation, or even have to take away the tape that has been placed and implant a new tape. Those events may bring troubles and heavy burden tooth medical staff and patients.

In addition, the present sling surgery has high requirements in screening appropriate urinary incontinence by preoperative assessment. This narrows the range of indications of urinary incontinence. As a result, for many types of stress urinary incontinence, there may be no chance of treatment by the tape surgery. For example, it is generally believed that the tape surgery is not applicable to stress urinary incontinence accompanied with weak contractility of the urethral sphincter muscle, i.e., type-III stress urinary incontinence. Due to the postoperative non-adjustability of the tape, the suspension of the tape in one direction cannot promise good urinary continence in most cases, because the present suspension in one direction can support only passive correction and the optimization to the combination of overall strength and elasticity is limited in such suspension way. Considering the postoperative non-adjustability of the tape, if the tape is excessively suspended in one direction, it may be less elastic. This may seriously affect the patient's micturition, resulting in dysuria.

The tape surgery is also not applicable to complex stress urinary incontinence accompanied with weak contractility of the bladder detrusor muscle. Suspension of the tape may increase the resistance of the urethra, leading to difficulty in emptying the bladder of the patient, thus causing and aggravating the condition of residual urine after micturition. If not corrected in time, this may seriously affect the function of the damaged bladder. Excessive aggravation of the condition of residual urine may induce overflow urinary incontinence. The long-term condition of large residual urine volume may greatly increase the risk of infections and stones, or even affect the function of the upper urinary tract and kidney, which may be life-threatening. Therefore, the present sling surgery is generally not recommended for patients who are found, before the surgery, with a large residual urine volume in the bladder and weak contractility of the bladder detrusor muscle. Consequently, the application of the present non-adjustable tape, which is suspended in one direction, in treating stress urinary incontinence is greatly limited.

### SUMMARY OF THE PRESENT INVENTION

### Technical Problems

The common tapes work well in treating surgically appropriate urinary incontinence in short and even medium term, with slight difference depending upon the types of the used tapes, treatment methods, and surgical approaches. The urinary continence capability after surgery can be improved by 80% or more. However, with the development of stress urinary incontinence and due to the non-adjustability of the tape after one-time implantation, urinary incontinence may recur several years later, even in the normal state without surgical complications and accidents, sometimes even back to the preoperative state. In the case of recurrence of urinary incontinence, many patients may be treated by the tape surgery again, hoping to restore urinary continence. However, due to the presence of the old tape and the local postoperative conditions, it is more risky and difficult to implant a new tape, and the postoperative effect is generally not as good as the first implantation. In addition, short-term postoperative complications may be significantly increased, especially rare and severe complications. The second implantation of a tape may increase the burden on both material and metal of patients.

### Technical Solutions

To solve the shortcomings such as the presence of multiple or single incision, suspension in one direction, physical lack of elasticity, and postoperative non-adjustability in clinic practice, and to widen the range of indications of stress urinary incontinence so that there is a probability of improving or even healing more types of urinary incontinence, especially those types of urinary incontinence that are considered not suitable for surgery, the present invention provides a novel tape system that is implanted by punching an external guide sheath. The present invention has the following advantages: there is no incision; the urethra is suspended crosswise in two directions, and the tape can be adaptively adjusted after operation according to the urinary continence condition and the state of urine storage and micturition functions of the bladder. Thus, it is a novel urethra tape system that can meet patients' individual and precise urine continence needs. By this tape, more patients with severe urine incontinence can be provided with good and controllable urine continence measures, the recurrence and development of urine incontinence can be delayed or even avoided, urine incontinence associated complications can be reduced, the burden on both individuals and the society can be reduced, and the quality of life of patients can be improved, or even patients can return back to the society.

The technical solution of the present invention will be described below. A novel incision-free adaptive TVT-XO for treatment of urinary incontinence is provided, comprising a tape body, wherein the tape body comprises a left tape and a right ape; each of the left tape and the right ape comprises a mesh band; a fixing anchor, configured to fix one end of the tape body between the obturator fascia and the obturator externus muscle of the pelvis, is disposed at one end of the mesh band; male and female snap buckles are disposed at the other ends of the mesh bands of both the left tape and the right ape, respectively; the left tape and the right ape are combined by means of insertion of the male and female snap buckles to form a cross structure, which is “ [symbol] ”-shaped.

A thin band, one end of which is connected to the male and female snap buckles and the other end of which is sewn to the mesh band, is further disposed between the male and female snap buckles and the mesh band.

An adjustment mechanism is further disposed in the middle of the thin band; the adjustment mechanism comprises a housing and a pulley located inside the housing; and the thin band is wound on the pulley and the length of the thin band out of the housing is controlled by the pulley.

A micro-motor and a control mechanism are further disposed in the adjustment mechanism; and the control mechanism controls the operation of the micro-motor to drive the pulley to rotate, in order to adjust the length of the thin band.

An opening is formed at each of two ends of the housing; and, the thin band is wound on the pulley, and two ends of the thin band extend out of the housing from the openings formed at the two ends of the housing.

A pressure sensor is further disposed on the surface of a side of the housing; and the pressure sensor receives the change in abdominal pressure and sends a signal to the control mechanism, to control the micro-motor to start or stop, in order to adjust the tightness of the tape.

The tape further comprises an external control switch; a wireless external program-controlled module is further disposed in the housing; and the wireless external program-controlled module receives a control signal sent by the external control switch, to control the micro-motor to start or stop by the control mechanism, in order to adjust the tightness of the tape.

The tape body and the thin band are both made of polymer synthetic materials.

Beneficial Effects

The present invention provides a novel incision-free adaptive TVT-XO for treatment of urinary incontinence, which is mainly applied in treating urinary incontinence to realize adaptive urinary continence after operation. By this tape, the optimal urinary continence is realized; the range of indications of urinary incontinence is widened, especially those types of urinary incontinence accompanied with severe bladder problems that are considered not suitable for surgery; the recurrence of urine incontinence is delayed and reduced; the urethra is suspended crosswise in two directions; and the tape can be adaptively adjusted after operation according to the urinary continence condition and the state of urine storage and micturition functions of the bladder. Thus, it is a novel urethra tape system that can meet patients' individual and precise urine continence needs. By this tape, more patients with severe urine incontinence can be provided with good urine continence measures, the recurrence and development of urine incontinence can be delayed or even avoided, urine incontinence associated complications can be reduced, the burden on both individuals and the society can be reduced, and the quality of life of patients can be improved, or even patients can return back to the society.

### BRIEF DESCRIPTION OF THE DRAWINGS

FIG. 1 is a structure diagram of the left tape according to the present invention;

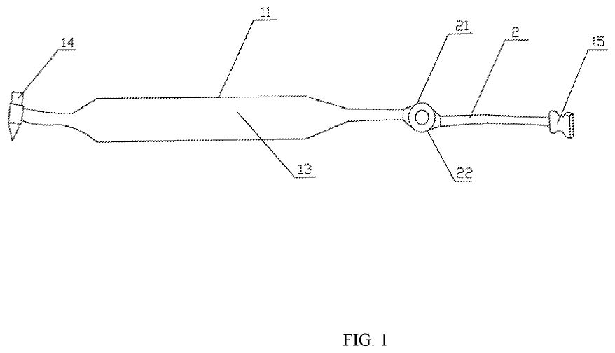

FIG. 2 is a structure diagram of the right ape according to the present invention;

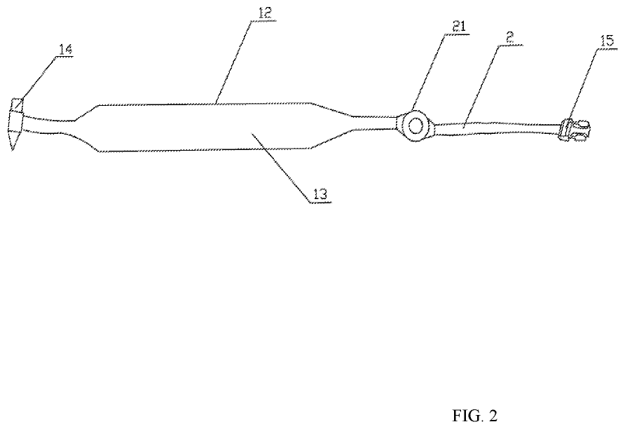

FIG. 3 a view showing the “ [symbol] ”-shaped cross structure, when the TVT-XO of the present invention is used,

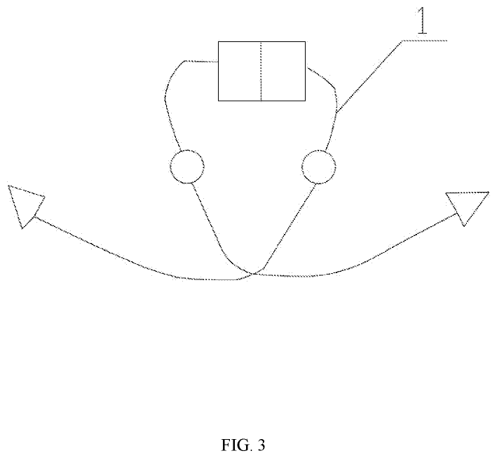

in which:

1: tape body; 2: thin band; 11: left tape; 12: right ape; 13: mesh band; 14: fixing anchor; 15: male and female snap buckles; 21: adjustment mechanism; 22: housing.

### DETAILED DESCRIPTION OF THE PRESENT INVENTION

The present invention will be further described below with reference to the accompanying drawings. The present invention is not limited to the embodiments to be described below.

A novel incision-free adaptive TVT-XO for treatment of urinary incontinence is provided, comprising a tape body (1), wherein the tape body (1) comprises a left tape (11) and a right ape (12); each of the left tape (11) and the right ape (12) comprises a mesh band (13); a fixing anchor (14), configured to fix one end of the tape body (1) between the obturator fascia and the obturator externus muscle of the pelvis, is disposed at one end of the mesh band (13); male and female snap buckles (15) are disposed at the other ends of the mesh bands (13) of both the left tape (11) and the right ape (120), respectively; the left tape (11) and the right ape (12) are combined by means of insertion of the male and female snap buckles (15) to form a cross structure, which is “ [symbol] ”-shaped. A thin band (2), one end of which is connected to the male and female snap buckles (15) and the other end of which is sewn to the mesh band (13), is further disposed between the male and female snap buckles (15) and the mesh band (13). In the present invention, the urethra is suspended crosswise in two directions, and the tape can be adaptively adjusted after operation according to the urinary continence condition and the state of urine storage and micturition functions of the bladder. Thus, it is a novel urethra tape system that can meet patients' individual and precise urine continence needs. By this tape, more patients with severe urine incontinence can be provided with good urine continence measures, the recurrence and development of urine incontinence can be delayed or even avoided, urine incontinence associated complications can be reduced, the burden on both individuals and the society can be reduced, and the quality of life of patients can be improved, or even patients can return back to the society.

An adjustment mechanism (21) is further disposed in the middle of the thin band (2); the adjustment mechanism (21) comprises a housing (22) and a pulley located inside the housing (22); and the thin band (2) is wound on the pulley and the length of the thin band (2) out of the housing (22) is controlled by the pulley. A micro-motor and a control mechanism are further disposed in the adjustment mechanism (21); and the control mechanism controls the operation of the micro-motor to drive the pulley to rotate, in order to adjust the length of the thin band (2). An opening is formed at each of two ends of the housing (22); and, the thin band (2) is wound on the pulley, and two ends of the thin band (2) extend out of the housing (22) from the openings formed at the two ends of the housing (22). A pressure sensor is further disposed on the surface of a side of the housing (22); and the pressure sensor receives the change in abdominal stress and sends a signal to the control mechanism, to control the micro-motor to start or stop, in order to adjust the tightness of the tape. The tape further comprises an external control switch; a wireless external program-controlled module is further disposed in the housing (22); and the wireless external program-controlled module receives a control signal sent by the external control switch that may be manually operated by a doctor or a patient, to control the micro-motor to start or stop by the control mechanism, in order to adjust the tightness of the tape. The tape body (1) and the thin band (2) are both made of polymer synthetic materials.

The foregoing description merely indicates preferred implementations of the present invention. The protection scope of the present invention is not limited to the above embodiments. All technical solutions based on the concept of the present invention shall be within the protection scope of the present invention. It should be noted that, for a person of ordinary skill in the art, various improvements and changes can be made to the present invention without departing from the principle of the present invention, and all those improvements and changes shall be considered as falling into the protection scope of the present invention.

What is claimed is:

1. A novel incision-free adaptive TVT-XO for treatment of urinary incontinence, comprising a tape body (1), wherein the tape body (1) comprises a left tape (11) and a right ape (12); each of the left tape (11) and the right ape (12) comprises a mesh band (13); a fixing anchor (14), configured to fix one end of the tape body (1) between the obturator fascia and the obturator externus muscle of the pelvis, is disposed at one end of the mesh band (13); male and female snap buckles (15) are disposed at the other ends of the mesh bands (13) of both the left tape (11) and the right ape (12), respectively; the left tape (11) and the right ape (12) are combined by means of insertion of the male and female snap buckles (15) to form a cross structure, which is “ [symbol] ”-shaped.

2. The novel incision-free adaptive TVT-XO for treatment of urinary incontinence according to claim 1, wherein a thin band (2), one end of which is connected to the male and female snap buckles (15) and the other end of which is sewn to the mesh band (13), is further disposed between the male and female snap buckles (15) and the mesh band (13).

3. The novel incision-free adaptive TVT-XO for treatment of urinary incontinence according to claim 2, wherein an adjustment mechanism (21) is further disposed in the middle of the thin band (2); the adjustment mechanism (21) comprises a housing (22) and a pulley located inside the housing (22); and the thin band (2) is wound on the pulley and the length of the thin band (2) out of the housing (22) is controlled by the pulley.

4. The novel incision-free adaptive TVT-XO for treatment of urinary incontinence according to claim 3, wherein a micro-motor and a control mechanism are further disposed in the adjustment mechanism (21); and the control mechanism controls the operation of the micro-motor to drive the pulley to rotate, in order to adjust the length of the thin band (2).

5. The novel incision-free adaptive TVT-XO for treatment of urinary incontinence according to claim 3, wherein an opening is formed at each of two ends of the housing (22); and, the thin band (2) is wound on the pulley, and two ends of the thin band (2) extend out of the housing (22) from the openings formed at the two ends of the housing (22).

6. The novel incision-free adaptive TVT-XO for treatment of urinary incontinence according to claim 4, wherein a pressure sensor is further disposed on the surface of a side of the housing (22); and the pressure sensor receives the change in abdominal stress and sends a signal to the control mechanism, to control the micro-motor to start or stop, in order to adjust the tightness of the tape.

7. The novel incision-free adaptive TVT-XO for treatment of urinary incontinence according to claim 3, wherein the tape further comprises an external control switch; a wireless external program-controlled module is further disposed in the housing (22); and the wireless external program-controlled module receives a control signal sent by the external control switch, to control the micro-motor to start or stop by the control mechanism, in order to adjust the tightness of the tape.

8. The novel incision-free adaptive TVT-XO for treatment of urinary incontinence according to claim 1, wherein the tape body (1) and the thin band (2) are both made of polymer synthetic materials.
